(19) United States
(12) Patent Application Publication
(10) Pub. No.: US 2008/0317829 A1
(43) Pub. Date: Dec. 25, 2008

(54) Method for the Production of a Wound Pad

(76) Inventors: Werner Michael Kulicke, Hamburg (DE); Tim Axel Wilhelms, Koblenz (DE)

Correspondence Address: AKERMAN SENTERFITT, P.O. BOX 3188, WEST PALM BEACH, FL, 33402-3188, US

(21) Appl. No.: 11/576,093

(22) PCT Filed: Sep. 23, 2005
(86) PCT No.: PCT/DE05/01686

§ 371 Date: Feb. 1, 2008

(30) Foreign Application Priority Data
Sep. 27, 2004 (DE) 10 2004 047 115.0

(51) Int. Cl. A61F 13/15

(52) U.S. Cl. 424/445

(57) ABSTRACT

The invention relates to a method for producing a wound pad, by means of which the permeability of the wound pad can be adjusted. The inventive method makes it possible to produce wound pads for a wide range of therapeutic applications.

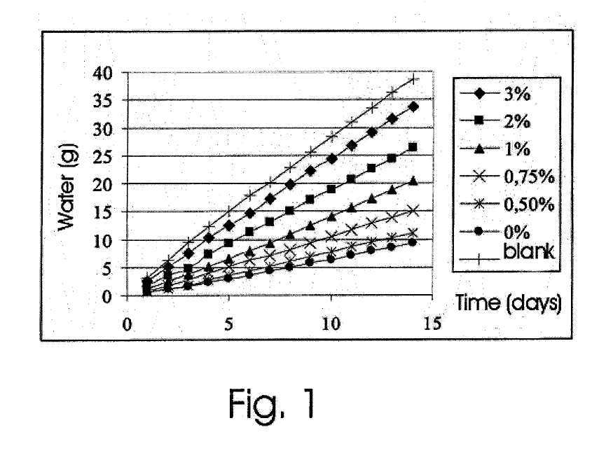

The present invention relates to a method for producing a wound dressing having a permeable polymer matrix.

Wound dressings play a role in the treatment of problem wounds such as burn wounds. A burn wound occurs due to local exposure to thermal energy or ionizing or UV radiation on the body surface. With severe burns, the skin is damaged in such deep layers that a skin graft is required. Treatment of the donor area (split-graft donor wound) has previously been performed in the operating room, i.e., immediately postoperatively, with cold and hot wrappings to achieve hemostasis. Then the wound is treated with ointment dressings. This results in high personnel costs because the wound must be cleaned and the bandages must be replaced every day. The ointment bandages that are used also adhere to the base of the wound, resulting in renewed traumatization of tissues when changing bandages. In addition, ointment dressings do not form a barrier for pathogens, so the latter can infect the wound from the outside. This also interferes with epithelialization of the wound.

Chronic wounds are another problem wound. This is a wound which does not show any tendency to heal after four to six weeks. The wound may be formed in a variety of ways. Chronic wounds or wounds with delayed healing usually involve an infection of the wound area. Chronic wounds encountered frequently include decubital ulcers and crural (leg) ulcers.

The variety of individual wounds also places special demands on the wound dressings. For wounds with a great deal of exudate such as burn wounds or split-graft donor wounds, the fluid coming from the wound must be absorbed and/or diverted, but a film of fluid should remain on the wound. For chronic wounds, i.e., wounds with little exudate, however, a lower permeability of the wound dressing is desirable.

DE 43 22 956 C2 describes a film of chitosan for sealing wounds; this film is to be used for sealing even skin abrasions, burns, scrapes, contusion wounds and defect wounds of a large extent and in particular chronic venous crural ulcers. The chitosan film described there has a perforated surface that promotes gas exchange, whereby the gas exchange is made possible essentially through breathing openings punched subsequently in the film. As the problem has presented itself, adequate respiration of the wound could not be ensured with a chitosan film of the type known until then, in particular in the case of sealing large area wounds. However, the film known from DE 43 22 956 C2 is difficult to manufacture, especially since the pores which are created mechanically must not exceed a certain size and smaller pores are very difficult to create. Furthermore, the barrier function of the membrane is destroyed by the perforations because the channels formed thereby extend through the entire membrane and allow unhindered access to the wound for microorganisms.

In addition, EP 0 927 053 B1 describes a fragmented polymer hydrogel which is used in particular to prevent tissue adhesions. This hydrogel may also have a plasticizer content of 0.1 to 30 percent by weight. However, the hydrogel is not a suitable wound dressing that promotes gas exchange. Moreover, the hydrogel does not have any pores.

DE 199 48 120 C2 describes a method for producing a biologically tolerable three-dimensional matrix with chitosan as the material for the matrix. The matrix has pores which are formed by freezing an aqueous chitosan solution and subliming the water under a reduced pressure. The pores serve to allow human or animal cells to grow into the matrix. However, due to the absence of a plasticizer, such a matrix does not ensure adequate gas exchange.

In addition, backing materials that contain block copolymers and plasticizers and can be used as wound dressings are also known from DE 197 29 905 A1. Backing materials of this type also have a certain air and water permeability, which, however, is not adjustable.

A membrane suitable for use as a wound closure is known from DE 690 29 969 T2. A plasticizer used in this membrane serves only to adjust the degree of elasticity.

Finally, the DE 39 28 858 A1 describes a hydrogel wound dressing with certain water vapor permeability values. However, this publication does not give specific information on adjusting the permeability values.

Porous membranes are also described in DE 100 50 870 A1 and in U.S. Pat. No. 5,993,661. The permeabilities of these membranes cannot be adjusted either.

The object of the present invention is therefore to provide a method by which wound dressings for various therapeutic applications can be produced.

According to this invention, the object formulated above is achieved by specifying a permeability of the wound dressing and using a plasticizer as a function of the selected permeability of the wound dressing, wherein the plasticizer is dissolved and the concentration of plasticizer in the solution is adjusted according to a correlation between the plasticizer concentration and the permeability determined for the particular polymer matrix and the particular plasticizer.

With the help of this method it is possible for the first time to utilize extensively the advantages of chitosan in wound healing, including hemostasis. Since different types of wounds also make different requirements of the permeability of wound dressings, the use of chitosan was very limited in the past.

In production of the wound dressing, the plasticizer may be added to a solution of the polymer and the polymer solution mixed with the plasticizer may then be processed further to form the wound dressing, preferably a polymer membrane in the form of a film. As an alternative to this, there is the option of producing a polymer membrane and then transferring this polymer membrane to the solution with the plasticizer. In both cases, the plasticizer results in an increase in the permeability of the wound dressing.

Especially stable and at the same time flexible wound dressings can be produced using chitosan as the polymer. The use of chitosan having a weight-average molecular weight of at least 100,000 g/mol has proven suitable here. Chitosan is essentially known. This is chitin that has been deacetylated to varying degrees. In a preferred process, a chitosan salt obtainable by dehydration of an acidic solution of chitosan is used.

According to a particular embodiment of the invention, the plasticizer is glycerol, a citric acid ester, glycerol triacetate, 1,2-propanediol or 1,2-propylene glycol. Likewise, polyethylene glycol (PEG) with a low molecular weight of up to 400 g/mol may also be used. The elasticity and flexibility of the membrane are decreased when PEG with a higher molecular weight is used. However, the present invention is not limited to the use of these plasticizers.

It has surprisingly been found that the water vapor permeability of the wound dressing can be increased by increasing the concentration of the plasticizer. Increasing the concentration of plasticizer in the polymer solution to 3.0 percent by weight (wt %) yields a membrane with which the polymer and the plasticizer are present in almost equal amounts. This is illustrated below on the example of chitosan/glycerol, reference being made here to the exemplary embodiments of the production of a chitosan membrane given further below.

| Polymer solution | Chitosan membrane |
|---|---|
| 0.25 wt % glycerol | 8 wt % glycerol |
| 0.50 wt % glycerol | 16 wt % glycerol |
| 0.75 wt % glycerol | 25 wt % glycerol |
| 1.00 wt % glycerol | 33 wt % glycerol |
| 2.00 wt % glycerol | 66 wt % glycerol |
| 3.00 wt % glycerol | 100 wt % glycerol |

100 percent by weight glycerol in the chitosan membrane means that there are equal weight ratios of glycerol and chitosan in the membrane. However, it must be assumed that glycerol can also be evaporated in the drawing process and thus the glycerol content in the membrane may be lower than the values given here.

Another advantage of the chitosan membrane is that the outer surface of the chitosan membrane remains elastic and dry after coming in contact with the moist wound. The side facing the wound develops a gelatinous consistency due to the partial adsorption of the wound secretion. This leads to cleansing of the wound and also to an adaptation of the chitosan membrane to irregularities in the wound surface. The physiologically important moist milieu of the wound is thereby retained during the exudative healing phase. Furthermore, the chitosan membrane sticks to the edge of the wound, so that no additional fixation of the membrane is required.

A wound dressing produced by the method according to this invention has a polymer matrix that contains a plasticizer and promotes gas exchange through the wound dressing, wherein the polymer matrix may have a pore structure that is obtainable by temporary inclusions into the polymer matrix. Such a wound dressing has the advantage in particular that the pores which are formed can be defined well in terms of size. For example, macropores may be formed by inclusions of silicate particles and micropores may be formed by inclusions of polyethylene glycol with a molecular weight of 1500 to 100,000 g/mol. Silicate particles and/or polyethylene glycol are then dissolved out of the polymer matrix. This makes it possible to adjust a pore size of 0.1 to 100 μm, for example. In addition, the surface structure of the wound dressing may play a role in wound healing, so it is also advantageous here to provide a certain pore structure.

Particularly, the wound dressing is a chitosan membrane. Such a membrane may have various thicknesses, depending on the area of application, e.g., 20 to 500 μm thick membranes may be used.

If special demands are made regarding the strength of the chitosan membrane, a crosslinking by means of epichlorohydrin or dialdehydes such as glutaraldehyde or glyoxal may also be performed.

On the other hand, the wound dressing may also be constructed in two or more layers, wherein the polymer matrix is provided with a backing material which may consist of synthetic or natural fibers. The backing material preferably comprises cellulose, viscose, polyamides, polyurethane, polyester, wool or cotton. The backing material ensures a wound dressing having a greater mechanical load bearing capacity.

### PRODUCTION EXAMPLES

1. Chitosan Membrane Containing Acid

Chitosan is dissolved in a dilute organic or inorganic acid, e.g., citric acid, tartaric acid, formic acid, hydrochloric acid, phosphoric acid and preferably lactic or acetic acid. To this solution is added a plasticizer such as glycerol in a concentration of 0.01 to 3%. This solution is applied to a glass plate or a plastic plate, in particular a plate of plexiglass or polycarbonate having a layer thickness of 1 to 10 mm. The plate coated with chitosan solution in this way is then dried at a temperature of 20 to 80° C. for several hours.

The membrane may then be removed easily from the synthesis plate. With regard to industrial production, polyester films may also be used as the backing. A winding or rolling technique may also be used.

2. Neutral Chitosan Membrane

Production is performed as described in Example 1, but the membrane is subsequently neutralized in an alkaline aqueous solution. Sodium hydroxide or potassium hydroxide solution may be used for this neutralization. The neutralized chitosan membrane is then placed for a few minutes in a 10% to 80% glycerol solution or in a 5% to 80% PEG solution. Finally, the membrane is dried for several hours at a temperature of 20° C. to 80° C.

3. Acidic Crosslinked Chitosan Membrane

A crosslinking agent such as glyoxal or glutaraldehyde is added to the chitosan solution in a ratio of 1/10 to 1/200 based on a basic unit of the polymer monomer. Otherwise, the procedure followed is the same as that described in Example 1.

4. Neutral Crosslinked Chitosan Membranes

In addition to the production according to Example 3, the membrane is subsequently neutralized in an alkaline aqueous solution. Then more plasticizer is added and the membrane is dried as described in Example 2.

5. Porous Chitosan Membrane

Silicate particles with a size of 5 to 60 μm are added to a chitosan solution in a concentration of 1% to 20%. After producing and drying the membrane, the silicate particles are dissolved out by adding sodium hydroxide solution. To do so, the chitosan membrane is placed in a 5% to 20% sodium hydroxide solution for one to three hours at 50° C. to 100° C. The porous chitosan membrane neutralized in this way is then placed in a 10% to 80% glycerol solution or in a 5% to 80% PEG solution and then dried again.

6. Porous Chitosan Membrane with Micropores

1% to 20% PEG having a molecular weight of 1500 to 100,000 g/mol is added to a chitosan solution. After production and drying of the membrane, the PEG is dissolved out in a dilute alkaline solution. To do so, the chitosan membrane is placed for 1 to 24 hours in a 0.1% to 3% sodium hydroxide solution at 20° C. to 60° C. The porous chitosan membrane neutralized in this way is then placed for a few minutes in a 10% to 80% glycerol solution or in a 5% to 80% PEG solution. The membrane is then dried for several hours at a temperature of 20° C. to 80° C.

7. Crosslinked Chitosan Membrane

The membrane is produced as described in Example 1. The membrane is then neutralized and crosslinked in an alkaline aqueous solution. To do so, the chitosan membrane is placed in a 1% to 5% sodium hydroxide solution that also contains epichlorohydrin as the crosslinking agent. The epichlorohydrin is added in a concentration of 0.000001 to 0.1 mol. The chitosan membrane is left in this solution for 1 to 5 hours at 20° C. to 80° C. . The chitosan membrane neutralized and crosslinked in this way is then placed for a few minutes in a 10% to 80% glycerol solution or in a 5% to 80% PEG solution. The membrane is then dried for several hours at a temperature of 20° C. to 80° C.

Determination of the Water Vapor Permeability of Chitosan Membranes

The water vapor permeability of a 70 μm thick chitosan membrane was determined as follows.

5 g CaCl₂ (anhydrous) was weighed into a 25 mL brown glass bottle. The opening of the bottle was sealed with a chitosan membrane. For the control, the same amount of CaCl₂ was placed in a second unsealed bottle. Both bottles were placed in a desiccator containing a saturated NaCl solution. The relative atmospheric humidity inside the desiccator was then adjusted to 75%. The water vapor permeability of the chitosan membrane was then determined each day for a period of 14 days by weighing the samples. The results for chitosan membranes with identical thickness and different glycerol contents of the membranes are shown in the figure. On day 14 of the test, the following water vapor permeability results were thus obtained:

| | Water vapor |
|---|---|
| Polymer solution | permeability |
| 0.00 wt % glycerol | 24.1% |
| 0.50 wt % glycerol | 28.9% |
| 0.75 wt % glycerol | 38.8% |
| 1.00 wt % glycerol | 52.6% |
| 2.00 wt % glycerol | 68.0% |
| 3.00 wt % glycerol | 86.9% |

Determination of Pore Structure

Pores of the polymer membranes can be measured by means of the essentially known mercury porosimetry (developed by Rittner and Drake in the year 1945) and by gas adsorption/desorption (BET, Langmuir).

Membranes produced according to Example 5 had macropores with a size of 60 μm, for example, corresponding to the size of the silicate particles used. The micropores produced by using PEG however were much smaller and had a pore radius of approximately 1 to 5 μm. With the macroporous membranes, the number of pores was proportional to the number of silicate particles used.

The roughness of the surface structures of the microporous or macroporous membranes varied. The side of the membrane facing the backing plate in the production of the membrane had a relatively smooth structure, but the side of the membrane facing away from the backing showed a rough spongy structure.

1. A method for producing a wound dressing having a permeable polymer matrix, wherein the permeability of the wound dressing is preselected and a plasticizer is used, depending on the selected permeability of the wound dressing, wherein the plasticizer is dissolved and the concentration of the plasticizer in the solution is adjusted according to a correlation between the plasticizer concentration and the permeability ascertained for the particular polymer matrix and the particular plasticizer.

2. The method according to claim 1, wherein the plasticizer is added to a solution of a polymer and the polymer solution mixed with the plasticizer is then processed further to produce the wound dressing.

3. The method according to claim 1, wherein a polymer membrane is produced and this polymer membrane is then transferred to the solution with the plasticizer.

4. The method according to claim 1, wherein the polymer is chitosan with a weight-average molecular weight of at least 100,000 g/mol.

5. The method according to claim 4, wherein instead of the chitosan, a chitosan salt obtainable by dehydrating an acidic solution of chitosan is used.

6. The method according to claim 1, wherein the plasticizer is glycerol, a citric acid ester, glycerol triacetate, 1,2-propanediol, 1,2-propylene glycol or polyethylene glycol with a low molecular weight of up to 400 g/mol.

7. The method according to claim 1, wherein the water vapor permeability of the wound dressing is increased with an increase in the concentration of the plasticizer.

8. The method according to claim 2, wherein the concentration of the plasticizer in the polymer solution is 0.01 to 3.0 percent by weight.

9. The method according to claim 8, wherein the polymer solution mixed with the plasticizer is processed further to form a polymer membrane in the form of a film.

10. The method according to claim 1, wherein the pore structure of the polymer matrix is adjusted.

11. The method according to claim 10, wherein particles are added to the polymer solution and then these particles are dissolved out of the polymer membrane after further processing the polymer solution to yield a polymer membrane.

12. The method according to claim 11, wherein the particles are silicate particles.

13. The method according to claim 10, wherein polyethylene glycol is added to the polymer solution and the polyethylene glycol is dissolved out of the polymer membrane after further processing of the polymer solution to form a polymer membrane.

14. The method according to claim 13, wherein polyethylene glycol with a molecular weight of 1500 to 100,000 g/mol is used.
